(19) United States
(12) Patent Application Publication
(10) Pub. No.: US 2010/0197744 A1
(43) Pub. Date: Aug. 5, 2010

(54) Organic Compounds

(76) Inventors: David Barnes, Cambridge, MA (US); Gary Mark Coppola, Cambridge, MA (US); Travis Stams, Cambridge, MA (US); Sidney Wolf Topiol, Cambridge, MA (US); James Richard Wareing, Cambridge, MA (US)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/602,709

(22) PCT Filed: Jun. 3, 2008
(86) PCT No.: PCT/EP2008/056807

§ 371 Date: Dec. 2, 2009

Related U.S. Application Data
(60) Provisional application No. 60/941,768, filed on Jun. 4, 2007.

(51) Int. Cl. A61K 31/433; C07D 285/08; A61P 3/10; A61P 13/12; A61P 9/06; A61P 9/12; A61P 9/10; A61P 3/06; A61P 35/00; A61P 19/10; A61P 25/16; A61P 25/28

(52) U.S. Cl. 514/361; 548/129

(57) ABSTRACT

Novel compounds that are inhibitors of protein tyrosine phosphatases (PTPases) and, thus, may be employed for the treatment of conditions mediated by PTPase activity. The compounds of the present invention may also be employed as inhibitors of other enzymes characterized with a phosphotyrosine binding region such as the SH2 domain. Accordingly, the compounds of the present invention may be employed for prevention and/or treatment of insulin resistance associated with obesity, glucose intolerance, diabetes mellitus, hypertension and ischemic diseases of the large and small blood vessels, conditions that accompany type-2 diabetes, including hyperlipidemia, hypertriglyceridemia, atherosclerosis, vascular restenosis, irritable bowel syndrome, pancreatitis, adipose cell tumors and carcinomas such as liposarcoma, dyslipidemia, and other disorders where insulin resistance is indicated. In addition, the compounds of the present invention may be employed to treat and/or prevent cancer, osteoporosis, neurodegenerative and infectious diseases, and diseases involving inflammation and the immune system.

The present invention relates to thiadiazolidinone derivatives, pharmaceutical compositions containing such compounds, methods of making such and methods of treating conditions mediated by protein tyrosine phosphatases by employing such compounds.

Accordingly, the present invention provides the following compounds:

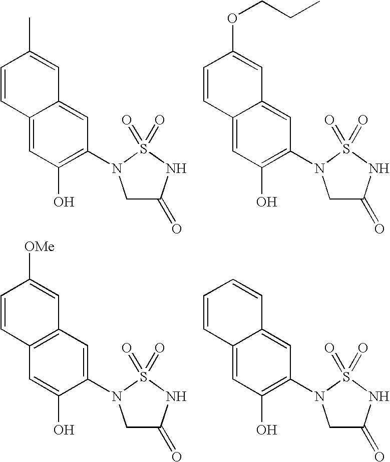

or a pharmaceutically acceptable salt thereof.

The compounds of the present invention are inhibitors of protein tyrosine phosphatases (PTPases); in particular, the compounds of the present invention inhibit PTPase-1B (PTP-1B) and T-cell PTPase (TC PTP) and, thus, may be employed for the treatment of conditions mediated by PTPase activity. Accordingly, the compounds of the present invention may be employed for treatment of insulin resistance, glucose intolerance, obesity, diabetes mellitus, hypertension and ischemic diseases of the large and small blood vessels, conditions accompanying type 2 diabetes including dyslipidemia, e.g., hyperlipidemia and hypertriglyceridemia, atherosclerosis, vascular restenosis, irritable bowel syndrome, pancreatitis, adipose cell tumors and carcinomas such as liposarcoma, dyslipidemia, and other disorders where insulin resistance is indicated. In addition, the compounds of the present invention may be employed to treat cancer, osteoporosis, neurodegenerative and infectious diseases, and diseases involving inflammation and the immune system.

The present invention also concerns the use of the compounds of the invention may be employed for treatment of insulin resistance, glucose intolerance, type 2 diabetes, renal insufficiency (diabetic and non-diabetic), diabetic nephropathy, glomerulonephritis, glomerular sclerosis, proteinuria of primary renal disease, diabetic retinopathy, obesity, all types of heart failures including acute and chronic congestive heart failure, left ventricular dysfunction and hypertrophic cardiomyopathy, diabetic cardiac myopathy, supraventricular and ventricular arrhythmias, atrial fibrillation and atrial flutter, hypertension, primary and secondary pulmonary hypertension, renal vascular hypertension, dyslipidemia, atherosclerosis, ischemic diseases of the large and small blood vessels, angina pectoris (whether unstable or stable), myocardial infarction and its sequelae, ischemia/reperfusion injury, detrimental vascular remodeling including vascular restenosis, management of other vascular disorders including migraine, peripheral vascular disease and Raynaud's disease, irritable bowel syndrome, pancreatitis, cancer, osteoporosis, multiple sclerosis, stroke, spinal cord injury, neurodegenerative diseases such as Alzheimer's, Parkinson's and polyglutamine disorders such as Huntington's and spinocerebellar ataxia, infectious diseases, and diseases involving inflammation and the immune system and diseases involving muscle degeneration.

Listed below are definitions of various terms used to describe the compounds of the instant invention. These definitions apply to the terms as they are used throughout the specification unless they are otherwise limited in specific instances either individually or as part of a larger group. In general, whenever an alkyl group is referred to as a part of the structure, an optionally substituted alkyl is also intended.

Pharmaceutically acceptable salts of any compound of the present invention refer to salts formed with bases, namely cationic salts such as alkali and alkaline earth metal salts, such as sodium, lithium, potassium, calcium, magnesium, as well as ammonium salts, such as ammonium, trimethylammonium, diethylammonium, and tris(hydroxymethyl)-methylammonium salts, and salts with amino acids.

As described herein above, the present invention provides 1,1-dioxo-1,2,5-thiadiazolidin-3-one derivatives, pharmaceutical compositions containing the same, methods for preparing such compounds and methods of treating and/or preventing conditions associated with PTPase activity, in particular, PTP-1B and TC PTP activity, by administration of a therapeutically effective amount of a compound of the present invention, or a pharmaceutical composition thereof.

Particular embodiments of the invention are:
5-(3-Hydroxy-7-methoxynaphthalen-2-yl)-1,1-dioxo-[1,2,5]thiadiazolidin-3-one;
5-(3-Hydroxy-7-methoxynaphthalen-2-yl)-1,1-dioxo-[1,2,5]thiadiazolidin-3-one potassium salt;
5-(3-Hydroxy-7-propoxynaphthalen-2-yl)-1,1-dioxo-[1,2,5]thiadiazolidin-3-one;
5-(3-Hydroxy-7-propoxynaphthalen-2-yl)-1,1-dioxo-[1,2,5]thiadiazolidin-3-one potassium salt;
5-(3-Hydroxynaphthalen-2-yl)-1,1-dioxo-[1,2,5]thiadiazolidin-3-one;
5-(3-Hydroxynaphthalen-2-yl)-1,1-dioxo-[1,2,5]thiadiazolidin-3-one potassium salt;
5-(3-Hydroxy-7-methyl-naphthalen-2-yl)-1,1-dioxo-1,2,5-thiadiazolidin-3-one; and
5-(3-Hydroxy-7-methyl-naphthalen-2-yl)-1,1-dioxo-1,2,5-thiadiazolidin-3-one potassium salt.

Compounds of the present invention can be made by using the following general procedure in Scheme 1.

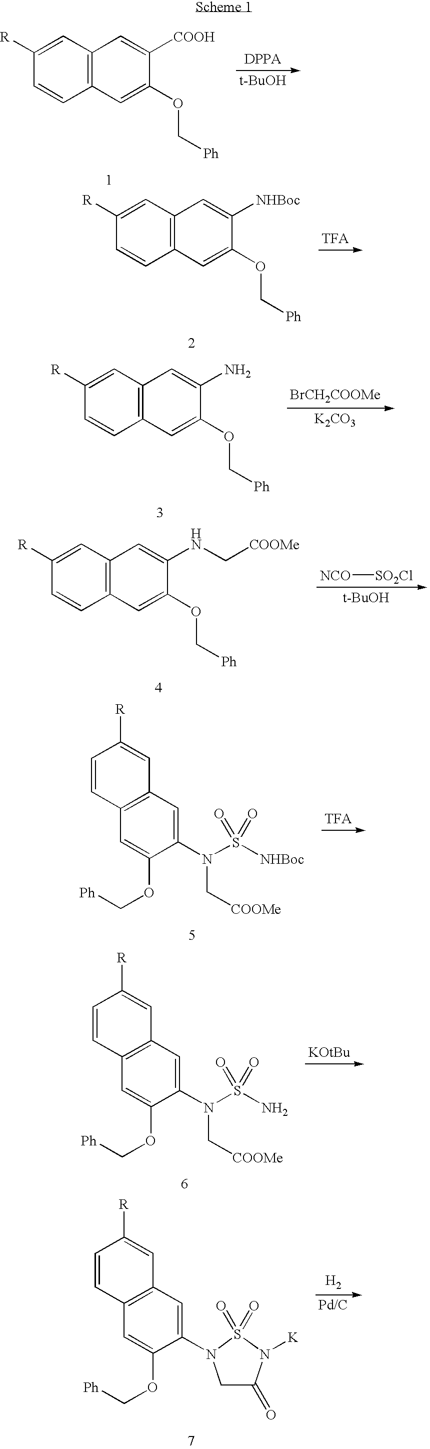

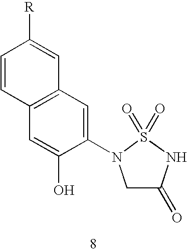

wherein R is hydrogen, C₁₋₄ alkyl, or C₁₋₄ alkoxy.
C₁₋₄ alkyl is defined as methyl, ethyl, propyl, isopropyl, butyl, iso-butyl, sec-butyl or t-butyl. C₁₋₄ alkoxy is defined as CH₃O—, CH₃CH₂O—, CH₃CH₂CH₂O—, (CH₃)₂CHO—, CH₃CH₂CH₂CH₂O—, (CH₃)₂CH₂CH₂O—, CH₃CH₂CH(CH₃)O—, OR(CH₃)₃CO—.

Compound 1 is converted from carboxylic acid to a protected amine compound 2. Trifluoroacetic acid (TFA) is used to remove the amino protecting group. The amino group of compound three is alkylated with an appropriated α-bromo ester. Compound 4 is converted to compound 5 by reacting chlorosulfonyl isocyanate with the appropriate alcohol in an organic solvent such as MeCN, DCM or THF.

TFA is used to remove the amino protecting group from compound 5. Compound 6 is cyclized to compound 7 is the presence of base. The hydroxyl protecting group is removed by hydrogenation to give compound 8.

The above mentioned reactions are carried out according to standard methods, in the presence or absence of diluent, preferably such as are inert to the reagents and are solvents thereof, of catalysts, condensing or said other agents respectively and/or inert atmospheres, at low temperatures, room temperature or elevated temperatures (preferably at or near the boiling point of the solvents used), and at atmospheric or super-atmospheric pressure. The preferred solvents, catalysts and reaction conditions are set forth in the appended illustrative Examples.

The invention further includes any variant of the present processes, in which an intermediate product obtainable at any stage thereof is used as starting material and the remaining steps are carried out, or in which the starting materials are formed in situ under the reaction conditions.

Compounds of the invention and intermediates can also be converted into each other according to methods generally known per se.

The invention also relates to any novel starting materials, intermediates and processes for their manufacture.

Finally, compounds of the invention are either obtained in the free form, or as prodrug derivatives thereof.

In particular, the NH-group of the 1,1-dioxo-1,2,5-thiadiazolidin-3-one moiety, may be converted into salts with pharmaceutically acceptable bases. Salts may be formed using conventional methods, advantageously in the presence of an ethereal or alcoholic solvent, such as a lower alkanol. From the solutions of the latter, the salts may be precipitated with ethers, e.g. diethyl ether. Resulting salts may be converted into the free compounds by treatment with acids. These or other salts can also be used for purification of the compounds obtained.

Prodrug derivatives of any compound of the present invention are derivatives of said compounds which following administration release the parent compound in vivo via some chemical or physiological process, e.g., a prodrug on being brought to the physiological pH or through enzyme action is converted to the parent compound. Exemplary prodrug derivatives are O-acyl derivatives of phenols, wherein acyl has a meaning as defined herein. Preferred are pharmaceutically acceptable ester derivatives convertible by hydrolysis under physiological conditions to the parent phenol.

In view of the close relationship between the free compounds, the prodrug derivatives and the compounds in the form of their salts, whenever a compound is referred to in this context, a prodrug derivative and a corresponding salt is also intended, provided such is possible or appropriate under the circumstances.

The compounds, including their salts, can also be obtained in the form of their hydrates, or include other solvents used for their crystallization.

As described herein above, the compounds of the present invention are inhibitors of PTPases and, thus, may be employed for the treatment of conditions mediated by the PTPases. Accordingly, the compounds of present invention may be employed for treatment of insulin resistance, glucose intolerance, obesity, diabetes mellitus, hypertension and ischemic diseases of the large and small blood vessels, conditions accompanying type 2 diabetes including dyslipidemia, e.g., hyperlipidemia and hypertriglyceridemia, atherosclerosis, vascular restenosis, irritable bowel syndrome, pancreatitis, adipose cell tumors and carcinomas such as liposarcoma, dyslipidemia, and other disorders where insulin resistance is indicated. In addition, the compounds of the present invention may be employed to treat cancer, osteoporosis, neurodegenerative and infectious diseases, and diseases involving inflammation and the immune system.

Accordingly, the compounds of the present invention may be employed for treatment of insulin resistance, glucose intolerance, type 2 diabetes, renal insufficiency (diabetic and non-diabetic), diabetic nephropathy, glomerulonephritis, glomerular sclerosis, proteinuria of primary renal disease, diabetic retinopathy, obesity, all types of heart failures including acute and chronic congestive heart failure, left ventricular dysfunction and hypertrophic cardiomyopathy, diabetic cardiac myopathy, supraventricular and ventricular arrhythmias, atrial fibrillation and atrial flutter, hypertension, primary and secondary pulmonary hypertension, renal vascular hypertension, dyslipidemia, atherosclerosis, ischemic diseases of the large and small blood vessels, angina pectoris (whether unstable or stable), myocardial infarction and its sequelae, ischemia/reperfusion injury, detrimental vascular remodeling including vascular restenosis, management of other vascular disorders including migraine, peripheral vascular disease and Raynaud's disease, irritable bowel syndrome, pancreatitis, cancer, osteoporosis, multiple sclerosis, stroke, spinal cord injury, neurodegenerative diseases such as Alzheimer's, Parkinson's and polyglutamine disorders such as Huntington's and spinocerebellar ataxia, infectious diseases, and diseases involving inflammation and the immune system and diseases involving muscle degeneration.

The present invention further provides pharmaceutical compositions comprising a therapeutically effective amount of a pharmacologically active compound of the instant invention, alone or in combination with one or more pharmaceutically acceptable carriers.

The pharmaceutical compositions according to the invention are those suitable for enteral, such as oral or rectal; transdermal and parenteral administration to mammals, including man, for the treatment of conditions mediated by PTPase activity, in particular, PTP-1B and TC PTP activity. Such conditions include e.g. insulin resistance, glucose intolerance, obesity, diabetes mellitus, hypertension and ischemic diseases of the large and small blood vessels, conditions accompanying type 2 diabetes including dyslipidemia, e.g., hyperlipidemia and hypertriglyceridemia, atherosclerosis, vascular restenosis, irritable bowel syndrome, pancreatitis, adipose cell tumors and carcinomas such as liposarcoma, dyslipidemia, and other disorders where insulin resistance is indicated. In addition, the compounds of the present invention may be employed to treat cancer, osteoporosis, neurodegenerative and infectious diseases, and diseases involving inflammation and the immune system.

Thus, the pharmacologically active compounds of the invention may be employed in the manufacture of pharmaceutical compositions comprising an effective amount thereof in conjunction or admixture with excipients or carriers suitable for either enteral or parenteral application. Preferred are tablets and gelatin capsules comprising the active ingredient together with:

a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and or polyvinylpyrrolidone; if desired
d) disintegrants, e.g., starches, agar, alginic acid or its sodium salt, or effervescent mixtures; and/or
e) absorbants, colorants, flavors and sweeteners. Injectable compositions are preferably aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, preferably about 1-50%, of the active ingredient.

Suitable formulations for transdermal application include a therapeutically effective amount of a compound of the invention with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Accordingly, the present invention provides pharmaceutical compositions as described above for the treatment of conditions mediated by PTPases, preferably, insulin resistance, glucose intolerance, obesity, diabetes mellitus, hypertension and ischemic diseases of the large and small blood vessels, conditions accompanying type 2 diabetes including dyslipidemia, e.g., hyperlipidemia and hypertriglyceridemia, atherosclerosis, vascular restenosis, irritable bowel syndrome, pancreatitis, adipose cell tumors and carcinomas such as liposarcoma, dyslipidemia, and other disorders where insulin resistance is indicated. In addition, the compounds of the present invention may be employed to treat cancer, osteoporosis, neurodegenerative and infectious diseases, and diseases involving inflammation and the immune system.

Accordingly, the present invention provides pharmaceutical compositions as described above for the treatment of conditions mediated by PTPases, preferably, insulin resistance, glucose intolerance, type 2 diabetes, renal insufficiency (diabetic and non-diabetic), diabetic nephropathy, glomerulonephritis, glomerular sclerosis, proteinuria of primary renal disease, diabetic retinopathy, obesity, all types of heart failures including acute and chronic congestive heart failure, left ventricular dysfunction and hypertrophic cardiomyopathy, diabetic cardiac myopathy, supraventricular and ventricular arrhythmias, atrial fibrillation and atrial flutter, hypertension, primary and secondary pulmonary hypertension, renal vascular hypertension, dyslipidemia, atherosclerosis, ischemic diseases of the large and small blood vessels, angina pectoris (whether unstable or stable), myocardial infarction and its sequelae, ischemia/reperfusion injury, detrimental vascular remodeling including vascular restenosis, management of other vascular disorders including migraine, peripheral vascular disease and Raynaud's disease, irritable bowel syndrome, pancreatitis, cancer, osteoporosis, multiple sclerosis, stroke, spinal cord injury, neurodegenerative diseases such as Alzheimer's, Parkinson's and polyglutamine disorders such as Huntington's and spinocerebellar ataxia, infectious diseases, and diseases involving inflammation and the immune system and diseases involving muscle degeneration.

The pharmaceutical compositions may contain a therapeutically effective amount of a compound of the invention as defined above, either alone or in a combination with another therapeutic agent, e.g., each at an effective therapeutic dose as reported in the art. Such therapeutic agents include:

a) anti-diabetic agents, such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; thiazolidone derivatives such as glitazones, e.g., pioglitazone and rosiglitazone; glucokinase activators; GSK3 (glycogen synthase kinase-3) inhibitors such as SB-517955, SB-4195052, SB-216763, N,N-57-05441 and N,N-57-05445; RXR ligands such as GW-0791 and AGN-194204; sodium-dependent glucose co-transporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; modulators of PPARs (peroxisome proliferator-activated receptors), e.g., non-glitazone type PPARγ agonists such as N-(2-benzoylphenyl)-L-tyrosine analogues, e.g. GI-262570, and JTT501; DPPIV (dipeptidyl peptidase IV) inhibitors such as LAF237, MK-0431, saxagliptin and GSK23A; SCD-1 (stearoyl-CoA desaturase-1) inhibitors; DGAT1 and DGAT2 (diacylglycerol acyltransferase 1 and 2) inhibitors; ACC2 (acetyl CoA carboxylase 2) inhibitors; and breakers of AGE (advanced glycation end products);
b) anti-dyslipidemic agents such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin; HDL increasing compounds such as cholesterol ester transfer protein (CETP) inhibitors, e.g., JTT705; Apo-A1 analogs and mimetics; squalene synthase inhibitors; FXR (farnesoid X receptor) and LXR (liver X receptor) ligands; cholestyramine; fibrates; nicotinic acid; and aspirin;
c) anti-obesity agents such as phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine, ecopipam, ephedrine, and pseudoephedrine; cholesterol absorption modulators such as ZETIA® and KT6-971; and cannabinoid receptor antagonists such as rimonabant; and
d) anti-hypertensive agents, e.g., loop diuretics such as ethacrynic acid, furosemide and torsemide; angiotensin converting enzyme (ACE) inhibitors such as benazepril, captopril, enalapril, fosinopril, lisinopril, moexipril, perinodopril, quinapril, ramipril and trandolapril; inhibitors of the Na-K-ATPase membrane pump such as digoxin; neutralendopeptidase (NEP) inhibitors; ACE/NEP inhibitors such as omapatrilat, sampatrilat and fasidotril; angiotensin II antagonists such as candesartan, eprosartan, irbesartan, losartan, telmisartan and valsartan, in particular valsartan; renin inhibitors such as ditekiren, zankiren, terlakiren, aliskiren, RO 66-1132 and RO-66-1168; β-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists such as eplerenone; and aldosterone synthase inhibitors such as anastrazole and fadrazole.

Other specific anti-diabetic compounds are described by Patel Mona in Expert Opin Investig Drugs, 2003, 12(4), 623-633, in the FIGS. 1 to 7, which are herein incorporated by reference. A compound of the present invention may be administered either simultaneously, before or after the other active ingredient, either separately by the same or different route of administration or together in the same pharmaceutical formulation.

The structure of the therapeutic agents identified by code numbers, generic or trade names may be taken from the actual edition of the standard compendium “The Merck Index” or from databases, e.g., Patents International (e.g. IMS World Publications). The corresponding content thereof is hereby incorporated by reference.

Accordingly, the present invention provides pharmaceutical compositions comprising a therapeutically effective amount of a compound of the invention in combination with a therapeutically effective amount of another therapeutic agent, preferably selected from anti-diabetics, hypolipidemic agents, anti-obesity agents or anti-hypertensive agents, most preferably from antidiabetics or anti-obesity agents as described above.

The present invention further relates to pharmaceutical compositions as described above for use as a medicament.

The present invention further relates to use of pharmaceutical compositions or combinations as described above for the preparation of a medicament for the treatment of conditions mediated by PTPase activity, in particular, PTP-1B and TC PTP activity. Such conditions include insulin resistance, glucose intolerance, obesity, diabetes mellitus, hypertension and ischemic diseases of the large and small blood vessels, conditions accompanying type 2 diabetes including dyslipidemia, e.g., hyperlipidemia and hypertriglyceridemia, atherosclerosis, vascular restenosis, irritable bowel syndrome, pancreatitis, adipose cell tumors and carcinomas such as liposarcoma, dyslipidemia, and other disorders where insulin resistance is indicated. In addition, the compounds of the present invention may be employed to treat cancer, osteoporosis, neurodegenerative and infectious diseases, and diseases involving inflammation and the immune system. Such conditions also include insulin resistance, glucose intolerance, type 2 diabetes, renal insufficiency (diabetic and non-diabetic), diabetic nephropathy, glomerulonephritis, glomerular sclerosis, proteinuria of primary renal disease, diabetic retinopathy, obesity, all types of heart failures including acute and chronic congestive heart failure, left ventricular dysfunction and hypertrophic cardiomyopathy, diabetic cardiac myopathy, supraventricular and ventricular arrhythmias, atrial fibrillation and atrial flutter, hypertension, primary and secondary pulmonary hypertension, renal vascular hypertension, dyslipidemia, atherosclerosis, ischemic diseases of the large and small blood vessels, angina pectoris (whether unstable or stable), myocardial infarction and its sequelae, ischemia/reperfusion injury, detrimental vascular remodeling including vascular restenosis, management of other vascular disorders including migraine, peripheral vascular disease and Raynaud's disease, irritable bowel syndrome, pancreatitis, cancer, osteoporosis, multiple sclerosis, stroke, spinal cord injury, neurodegenerative diseases such as Alzheimer's, Parkinson's and polyglutamine disorders such as Huntington's and spinocerebellar ataxia, infectious diseases, and diseases involving inflammation and the immune system and diseases involving muscle degeneration.

Thus, the present invention also relates to a compound of the present invention for use as a medicament, to the use of a compound of the present invention for the preparation of a pharmaceutical composition for treatment of conditions mediated by PTPase activity, in particular, PTP-1B and TC PTP activity, and to a pharmaceutical composition for use in conditions mediated by PTPase activity, in particular, PTP-1B and TC PTP activity, comprising a compound of the present invention, or a pharmaceutically acceptable salt thereof, in association with a pharmaceutically acceptable diluent or carrier therefore.

The present invention further provides a method for the treatment of conditions mediated by PTPase activity, in particular, PTP-1B and TC PTP activity, which method comprises administering a therapeutically effective amount of a compound of the present invention.

A unit dosage for a mammal of about 50 to 70 kg may contain between about 1 mg and 1000 mg, advantageously between about 5 mg to 500 mg of the active ingredient. The therapeutically effective dosage of a compound of formula I is dependent on the species of warm-blooded animal (mammal), the body weight, age and individual condition, on the form of administration, and on the compound involved.

In accordance with the foregoing the present invention also provides a therapeutic combination, e.g., a kit, kit of parts, e.g., for use in any method as defined herein, comprising a compound of the present invention, or a pharmaceutically acceptable salt thereof, to be used concomitantly or in sequence with at least one pharmaceutical composition comprising at least another therapeutic agent, preferably selected from anti-diabetic agents, hypolipidemic agents, anti-obesity agents or anti-hypertensive agents. The kit may comprise instructions for its administration.

Similarly, the present invention provides a kit of parts comprising: (i) a pharmaceutical composition of the invention; and (ii) a pharmaceutical composition comprising a compound selected from an anti-diabetic, a hypolipidemic agent, an anti-obesity agent, an anti-hypertensive agent, or a pharmaceutically acceptable salt thereof, in the form of two separate units of the components (i) to (ii).

Likewise, the present invention provides a method as defined above comprising co-administration, e.g., concomitantly or in sequence, of a therapeutically effective amount of a compound of the present invention, or a pharmaceutically acceptable salt thereof, and a second drug substance, said second drug substance being an anti-diabetic, a hypolipidemic agent, an anti-obesity agent or an anti-hypertensive agent, e.g., as indicated above.

Preferably, a compound of the invention is administered to a mammal in need thereof.

Preferably, a compound of the invention is used for the treatment of a disease which responds to modulation of PTPase activity, in particular, PTP-1B and TC PTP activity.

Preferably, the condition associated with PTPase activity, in particular, PTP-1B and TC PTP activity, is selected from insulin resistance, glucose intolerance, type 2 diabetes, renal insufficiency (diabetic and non-diabetic), diabetic nephropathy, glomerulonephritis, glomerular sclerosis, proteinuria of primary renal disease, diabetic retinopathy, obesity, all types of heart failures including acute and chronic congestive heart failure, left ventricular dysfunction and hypertrophic cardiomyopathy, diabetic cardiac myopathy, supraventricular and ventricular arrhythmias, atrial fibrillation and atrial flutter, hypertension, primary and secondary pulmonary hypertension, renal vascular hypertension, dyslipidemia, atherosclerosis, ischemic diseases of the large and small blood vessels, angina pectoris (whether unstable or stable), myocardial infarction and its sequelae, ischemia/reperfusion injury, detrimental vascular remodeling including vascular restenosis, management of other vascular disorders including migraine, peripheral vascular disease and Raynaud's disease, irritable bowel syndrome, pancreatitis, cancer, osteoporosis, multiple sclerosis, stroke, spinal cord injury, neurodegenerative diseases such as Alzheimer's, Parkinson's and polyglutamine disorders such as Huntington's and spinocerebellar ataxia, infectious diseases, and diseases involving inflammation and the immune system and diseases involving muscle degeneration.

Preferably, the condition associated with PTPase activity, in particular, PTP-1B and TC PTP activity, is selected from insulin resistance, glucose intolerance, obesity, diabetes mellitus, hypertension and ischemic diseases of the large and small blood vessels, conditions accompanying type 2 diabetes including dyslipidemia, e.g., hyperlipidemia and hypertriglyceridemia, atherosclerosis, vascular restenosis, irritable bowel syndrome, pancreatitis, adipose cell tumors and carcinomas such as liposarcoma, dyslipidemia, and other disorders where insulin resistance is indicated. In addition, the compounds of the present invention may be employed to treat cancer, osteoporosis, neurodegenerative and infectious diseases, and diseases involving inflammation and the immune system.

Finally, the present invention provides a method or use which comprises administering a compound of the present invention in combination with a therapeutically effective amount of an anti-diabetic agent, a hypolipidemic agent, an anti-obesity agent or an anti-hypertensive agent.

Ultimately, the present invention provides a method or use which comprises administering a compound of the present invention in the form of a pharmaceutical composition as described herein.

As used throughout the specification and in the claims, the term “treatment” embraces all the different forms or modes of treatment as known to those of the pertinent art and in particular includes preventive, curative, delay of progression and palliative treatment.

The above-cited properties are demonstrable in vitro and in vivo tests, using advantageously mammals, e.g., mice, rats, dogs, monkeys or isolated organs, tissues and preparations thereof. Said compounds can be applied in vitro in the form of solutions, e.g. preferably aqueous solutions, and in vivo either enterally, parenterally, advantageously intravenously, e.g. as a suspension or in aqueous solution. The dosage in vitro may range between about 1 μM and 1 nM concentrations, preferably between 10 nM and 200 nM. A therapeutically effective amount in vivo may range depending on the route of administration, between about 1 and 500 mg/kg, preferably between about 1 and 100 mg/kg.

The activity of a compound according to the invention may be assessed by the following methods or by following methods well described in the art (e.g. Peters G. et al. J. Biol. Chem., 2000, 275, 18201-09).

For example, the PTP-1B inhibitory activity in vitro may be determined as follows:

Assessment of human PTP-1B (hPTP-1B) activity in the presence of various agents is determined by measuring the amount of inorganic phosphate released from a phosphopeptide substrate using a 96-well microtiter plate format. The assay (100 μL) is performed in an assay buffer comprised of 50 mM TRIS (pH 7.5), 50 mM NaCl, 3 mM DTT at ambient temperature. The assay is typically performed in the presence of 0.4% dimethyl sulfoxide (DMSO). However, concentrations as high as 10% are used with certain poorly soluble compounds. A typical reaction is initiated by the addition of 0.4 pmoles of hPTP-1B (amino acids 1-411) to wells containing assay buffer, 3 nmoles of the synthetic phosphopeptide substrate (GNGDpYMPMSPKS), and the test compound. After 10 min, 180 μL malachite green reagent (0.88 mM malachite green, 8.2 mM ammonium molybdate, aqueous 1 N HCl, and 0.01% Triton X-100) is added to terminate the reaction. Inorganic phosphate, a product of the enzyme reaction, is quantitiated after 15 min as the green color resulting from complexing with the Malichite reagent and is determined as an A₆₂₀ using a Molecular Devices (Sunnyvale, Calif.) SpectraMAX Plus spectrophotometer. Test compounds are solubilized in 100% DMSO (Sigma, D-8779) and diluted in DMSO. Activity is defined as the net change in absorbance resulting from the activity of the uninhibited hPTP-1B_{[1-411]} minus that of a tube with acid-inactivated hPTP-1B_{[1-411]}.

The hPTP-1B_{[1-411]} is cloned by PCR from a human hippocampal cDNA library (Clonetech) and inserted into a pET 19-b vector (Novagen) at the Nco1 restriction site. E. coli strain BL21 (DE3) is transformed with this clone and stored as a stock culture in 20% glycerol at −80° C. For enzyme production, a stock culture is inoculated into Lb/Amp and grown at 37° C. Expression of PTP-1B is initiated by induction with 1 mM IPTG after the culture had reached an OD₆₀₀=0.6. After 4 h, the bacterial pellet is collected by centrifugation. Cells are resuspended in 70 mL lysis buffer (50 mM Tris, 100 mM NaCl, 5 mM DTT, 0.1% Triton X-100, pH7.6), incubated on ice for 30 min then sonicated (4×10 sec bursts at full power). The lysate is centrifuged at 100,000×g for 60 min and the supernatant is buffer exchanged and purified on a cation exchange POROS 20SP column followed by an anion exchange Source 30Q (Pharmacia) column, using linear NaCl gradient elutions. Enzyme is pooled, adjusted to 1 mg/mL and frozen at ±80° C.

Alternatively, the assessment of human PTP-1B activity in the presence of various agents may be determined by measuring the hydrolysis products of known competing substrates. For example, cleavage of substrate para-nitrophenylphosphate (pNPP) results in the release of the yellow-colored para-nitrophenol (pNP) which can be monitored in real time using a spectrophotometer. Likewise, the hydrolysis of the fluorogenic substrate 6,8-difluoro-4-methylumbelliferyl phosphate ammonium salt (DiFMUP) results in the release of the fluorescent DiFMU which can be readily followed in a continuous mode with a fluorescence reader (Anal. Biochem. 273, 41, 1999; Anal. Biochem. 338, 32, 2005):

pNPP Assay

Compounds were incubated with 1 nM recombinant human PTP-1B_{[1-298]} or PTP-1B_{[1-322]} in buffer (50 mM Hepes, pH 7.0, 50 mM KCl, 1 mM EDTA, 3 mM DTT, 0.05% NP-40 for 5 min at room temperature. The reaction is initiated by the addition of pNPP (2 mM final concentration) and run for 120 min at room temperature. Reactions are quenched with 5 N NaOH. Absorbance at 405 nm is measured using any standard 384 well plate reader.

### DiFMUP Assay

Compounds are incubated with 1 nM recombinant human PTP-1B_{[1-298}] or PTP-1B_{[1-322]} in buffer (50 mM Hepes, pH 7.0, 50 mM KCl, 1 mM EDTA, 3 mM DTT, 0.05% NP-40 (or 0.001% BSA) for 5 min at room temperature. The reaction is initiated by the addition of DiFMUP (6 μM final concentration) and run kinetically on fluorescence plate reader at 355 nm excitation and 460 nm emission wavelengths. Reaction rates over 15 min are used to calculate inhibition.

PTP-1B_{[1.298]} is expressed in E. coli BL21(DE3) containing plasmids constructed using pET19b vectors (Novagen). The bacteria are grown in minimal media using an “On Demand” Fed-batch strategy. Typically, a 5.5 liter fermentation is initiated in Fed-batch mode and grown overnight unattended at 37° C. Optical densities varied between 20-24 OD₆₀₀ and the cultures are induced at 30° C. with IPTG to a final concentration of 0.5 mM. The bacterial cells are harvested 8 hours later and yield 200-350 gm (wet weight). The cells are frozen as pellets and stored at −80° C. until use. All steps are performed at 4° C. unless noted. Cells (˜15 g) are thawed briefly at 37° C. and resuspended in 50 mL of lysis buffer containing 50 mM Tris-HCl, 150 mM NaCl, 5 mM DTT, pH 8.0 containing one tablet of Complete (EDTA-free) protease cocktail (Boehringer Mannheim), 100 μM PMSF and 100 μg/mL DNase I. The cells are lysed by sonication (4×10 second burst, full power) using a Virsonic 60 (Virtus). The pellet is collected at 35,000×g, resuspended in 25 mL of lysis buffer using a Polytron and collected as before. The two supernatants are combined and centrifuged for 30 min at 100,000×g. The soluble lysate could be stored at this stage at −80° C. or used for further purification. Diafiltration using a 10 kD MWCO membrane is used to buffer exchange the protein and reduce the NaCl concentration prior to cation exchange chromatography. Diafiltration buffer contained 50 mM MES, 75 mM NaCl, 5 mM DTT, pH 6.5. Soluble supernatant is then loaded onto a POROS 20 SP (1×10 cm) column equilibrated with cation exchange buffer (50 mM MES and 75 mM NaCl, pH 6.5) at a rate of 20 mL/min. An analytical column (4.6×100 mm) is run in a similar fashion except the flow rate was reduced to 10 mL/min. Protein is eluted from the column using a linear salt gradient (75-500 mM NaCl in 25 CV). Fractions containing PTP-1B_{[1-298]} are identified and pooled according to SDS-PAGE analyses. Final purification is performed using Sephacryl S-100 HR (Pharmacia). The column (2.6×35 cm) is equilibrated with 50 mM HEPES, 100 mM NaCl, 3 mM DTT, pH 7.5 and run at a flow rate of 2 mL/min. The final protein is pooled and concentrated to ˜5 mg/mL using an Ultrafree-15 concentrator (Millipore) with a MWCO 10,000. The concentrated protein is stored at −80° C. until use.

Competitive binding to the active site of the enzyme may be determined as follows:

Ligand binding is detected by acquiring ¹H-¹⁵N HSQC spectra on 250 μL of 0.15 mM PTP-1B_{[1-298]} in the presence and absence of added compound (1-2 mM). The binding is determined by the observation of ¹⁵N- or ¹H-amide chemical shift changes in two dimensional HSQC spectra upon the addition of a compound to ¹⁵N-label protein. Because of the ¹⁵N spectral editing, no signal from the ligand is observed, only protein signals. Thus, binding can be detected at high compound concentrations. Compounds which caused a pattern of chemical shift changes similar to the changes seen with known active site binders are considered positive.

All proteins are expressed in E. coli BL21 (DE3) containing plasmids constructed using pET19b vectors (Novagen). Uniformly ¹⁵N-labeled PTP-1B₁₋₂₉₈ is produced by growth of bacteria on minimal media containing ¹⁵N-labeled ammonium chloride. All purification steps are performed at 4° C. Cells (˜15 g) are thawed briefly at 37° C. and resuspended in 50 mL of lysis buffer containing 50 mM Tris-HCl, 150 mM NaCl, 5 mM DTT, pH 8.0 containing one tablet of Complete (EDTA-free) protease cocktail (Boehringer Mannheim), 100 μM PMSF and 100 μg/mL DNase I. The cells are lysed by sonication. The pellet is collected at 35,000×g, resuspended in 25 mL of lysis buffer using a Polytron and collected as before. The two supernatants are combined and centrifuged for 30 min at 100,000×g. Diafiltration using a 10 kD MWCO membrane is used to buffer exchange the protein and reduce the NaCl concentration prior to cation exchange chromatography. Diafiltration buffer contained 50 mM MES, 75 mM NaCl, 5 mM DTT, pH 6.5. Soluble supernatant is then loaded onto a POROS 20 SP (1×10 cm) column equilibrated with cation exchange buffer (50 mM MES and 75 mM NaCl, pH 6.5) at a rate of 20 mL/min. Protein is eluted from the column using a linear salt gradient (75-500 mM NaCl in 25 CV). Fractions containing PTP-1B's are identified and pooled according to SDS-PAGE analyses. PTP-1B₁₋₂₉₈ is further purified by anion exchange chromatography using a POROS 20 HQ column (1×10 cm). The pool from cation exchange chromatography is concentrated and buffer exchanged in 50 mM Tris-HCl, pH 7.5 containing 75 mM NaCl and 5 mM DTT. Protein is loaded onto column at 20 mL/min and eluted using a linear NaCl gradient (75-500 mM in 25 CV). Final purification is performed using Sephacryl S-100 HR (Pharmacia)(50 mM HEPES, 100 mM NaCl, 3 mM DTT, pH 7.5). The NMR samples are composed of uniformly ¹⁵N-labeled PTP-1B₁₋₂₉₈ (0.15 mM) and inhibitor (1-2 mM) in a 10% D₂O/90% H₂O Bis-Tris-d₁₉ buffer (50 mM, pH=6.5) solution containing NaCl (50 mM), DL-1,4-Dithiothreitol-d₁₀ (5 mM) and Sodium azide (0.02%).

The ¹H-¹⁵N HSQC NMR spectra are recorded at 20° C., on Bruker DRX500 or DMX600 NMR spectrometers. In all NMR experiments, pulsed field gradients are applied to afford the suppression of solvent signal. Quadrature detection in the indirectly detected dimensions is accomplished by using the States-TPPI method. The data are processed using Bruker software and analyzed using NMRCompass software (MSI) on Silicon Graphics computers.

The glucose and insulin lowering activity in vivo may be evaluated as follows:

Adult male C57BL ob/ob mice (Jackson Lab, Bar Harbor, Me.) at the age of 11 weeks are housed six per cage in a reversed light cycle room (light on from 6:00 p.m. to 6:00 a.m.) and given access to Purina rodent chow and water ad libitum. On day 1 tail blood samples are taken at 8:00 am and plasma glucose levels are determined. The animals are randomly assigned to the control and compound groups. The means of plasma glucose values of the groups are matched. Animals are then orally dosed with vehicle (0.5% carboxymethyl-cellulose with 0.2% Tween-80) or compounds (at 30 mg/kg) in vehicle. The mice are dosed daily for a total of 3 days. On day 4 basal blood samples are taken. The plasma samples are analyzed for glucose concentrations using a YSI2700 Dual Channel Biochemistry Analyzer (Yellow Springs Instrument Co., Yellow Springs, Ohio) and insulin concentrations using an ELISA assay.

The present invention includes all pharmaceutically acceptable isotopically-labeled compounds of formula (I) wherein one or more atoms are replaced by atoms having the same atomic number, but an atomic mass or mass number different from the atomic mass or mass number usually found in nature.

Examples of isotopes suitable for inclusion in the compounds of the invention comprises isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Isotopically-labeled compounds of formula (I) can generally be prepared by conventional techniques known to those skilled in the art or by processes analogous to those described in the accompanying Examples and Preparations Sections using an appropriate isotopically-labeled reagent in place of the non-labeled reagent previously employed.

The following Examples are intended to illustrate the invention and are not to be construed as being limitations thereon. Temperatures are given in degrees Centrigrade (° C.). If not mentioned otherwise, all evaporations are performed under reduced pressure, preferably between about 15 and 100 mmHg (=20-133 mbar). The structure of final products, intermediates and starting materials is confirmed by standard analytical methods, e.g. microanalysis, melting point (mp) and spectroscopic characteristics (e.g. MS, IR, NMR). In general, abbreviations used are those conventional in the art.

### EXAMPLE 1

### 5-(3-Hydroxy-7-methoxynaphthalen-2-yl)-1,1-dioxo-[1,2,5]thiadiazolidin-3-one potassium salt

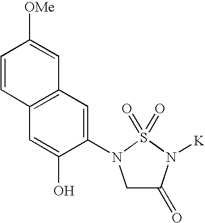

### Step 1

### 3-Benzyloxy-7-methoxynaphthalene-2-carboxylic acid benzyl ester

A mixture of 5.0 g (21.7 mmol) of 3-hydroxy-7-methoxynaphthalene-2-carboxylic acid, 9.29 g (54.3 mmol) of benzyl bromide, and 9.01 g (65.2 mmol) of potassium carbonate in 25 mL of DMF is stirred at 60° C. for 24 h. After allowing to cool to room temperature the mixture is poured into water and is extracted into EtOAc. The organic phase is washed 3× with water and 1× with saturated NaCl. The organic phase is dried over sodium sulfate and the solvent is removed under reduced pressure. The residual solid is crystallized from methylene chloride/ethanol to give the title compound as a tan solid, mp 96-98°. ¹H-NMR (CDCl₃): δ 8.25 (s, 1H), 7.61 (d, J=8.83 Hz, 1H), 7.49 (d, J=7.35 Hz, 2H), 7.46-7.30 (m, 7H), 7.24 (d, J=5.88 Hz, 2H), 7.18 (dd, J=9.20 and 2.58 Hz, 1H), 7.12 (m, 1H), 5.40 (s, 2H), 5.23 (s, 2H), 3.89 (s, 3H). Anal. Calcd for C₂₆H₂₂O₄: C, 78.38; H, 5.57. Found: C, 78.28; H, 5.56.

### Step 2

### 3-Benzyloxy-7-methoxynaphthalene-2-carboxylic acid

To a suspension of 6.07 g (20 mmol) of 3-benzyloxy-7-methoxynaphthalene-2-carboxylic acid benzyl ester in 100 mL of ethanol is added 24 mL of 1.0N NaOH (1.2 equiv) and the mixture is stirred at 60° C. for 4 h and at room temperature 16 h. The solvent is removed under reduced pressure and the residual solid is dissolved in 50 mL of water. The solution is washed with ether and the aqueous phase is acidified with 2N HCl. The resulting precipitate is filtered, washed with water and dried to give the title compound as a pale-yellow solid, mp 177-179°. ¹H-NMR (CDCl₃): δ 11.01 (s, broad, 1H), 8.70 (s, 1H), 7.67 (d, J=8.82 Hz, 1H), 7.53-7.41 (m, 5H), 7.37 (s, 1H), 7.27 (dd, J=8.83 and 2.57 Hz, 1H), 7.20 (m, 1H), 5.36 (s, 2H), 3.92 (s, 3H). Anal. Calcd for C₁₉H₁₆O₄: C, 74.01; H, 5.23. Found: C, 73.71; H, 5.46.

### Step 3

### (3-Benzyloxy-7-methoxynaphthalen-2-yl)-carbamic acid tert-butyl ester

To a suspension of 4.6 g (14.9 mmol) 3-benzyloxy-7-methoxynaphthalene-2-carboxylic acid in 40 mL of anhydrous t-BuOH+40 mL of anhydrous toluene is added 2.3 g (22 mmol) of triethylamine. To the resulting solution is added 5.34 g (19.4 mmol) of DPPA and the mixture is stirred at room temperature for 5 min then at 100° C. for 18 h. The mixture is allowed to cool to room temperature then is poured into water. The mixture is extracted into EtOAc and the organic phase is washed with saturated NaCl. The solvent is removed under reduced pressure and the residue is purified by flash chromatography using hexane/ethyl acetate (85:15) to elute the title compound as an oil which slowly becomes a waxy solid. ¹H-NMR (CDCl₃): δ 8.48 (s, 1H), 7.50 (d, J=8.82 Hz, 1H), 7.48-7.31 (m, 7H), 7.10 (s, 1H), 6.99 (dd, J=8.82 and 2.57 Hz, 1H), 5.17 (s, 2H), 3.84 (s, 3H), 1.54 (s, 9H).

### Step 4

### 3-Benzyloxy-7-methoxynaphthalen-2-ylamine

A solution of 4.99 g (13.1 mmol) of (3-benzyloxy-7-methoxynaphthalen-2-yl)-carbamic acid tert-butyl ester in 50 mL of TFA/methylene chloride (1:1) is stirred at room temperature for 30 min. The solvent is removed under reduced pressure and 10% aqueous sodium bicarbonate is added to the residue. The mixture is extracted into EtOAc and the organic phase is dried over sodium sulfate. The solvent is removed under reduced pressure and the residue is purified by flash chromatography using methylene chloride to elute the title compound which is crystallized from ethanol to give a white solid, mp 149-151°. ¹H-NMR (CDCl₃): δ 7.54-7.32 (m, 6H), 7.09 (s, 1H), 6.92 (d, J=9.20 Hz, 2H), 6.89 (dd, J=8.82 and 2.57 Hz, 1H), 5.18 (s, 2H), 4.11 (s, broad, 2H), 3.87 (s, 3H).

### Step 5

### (3-Benzyloxy-7-methoxynaphthalen-2-ylamino)-acetic acid methyl ester

To a mixture of 2.76 g (9.88 mmol) of 3-benzyloxy-7-methoxynaphthalen-2-ylamine and 2.73 g (19.8 mmol) of potassium carbonate in 20 mL of DMF is added 2.26 g (14.8 mmol) of methyl bromoacetate. The mixture is stirred at 60° C. for 2 h then at room temperature for 16 h. The mixture is poured into water and extracted into EtOAc. The organic phase is washed with water (3×), sat. NaCl (1×), and is dried over sodium sulfate. The solvent is removed under reduced pressure and the residue is crystallized from ethanol to give the title compound as an off-white solid, mp 129-131°.; NMR (CDCl₃): δ 7.50 (s, broad, 2H), 7.49 (d, J=8.46 Hz, 1H), 7.45-7.33 (m, 3H), 7.07 (s, 1H), 6.97 (d, J=2.20 Hz, 1H), 6.89 (dd, J=8.82 and 2.57 Hz, 1H), 6.62 (s, 1H), 5.20 (s, 3H), 4.05 (s, 2H), 3.88 (s, 3H), 3.80 (s, 3H). MS (M+1): 352. Anal. Calcd for C₂₁H₂₁NO₄: C, 71.78; H, 6.02; N, 3.99. Found: C, 71.61; H, 5.86; N, 3.91.

### Step 6

### N-(t-Butoxycarbonylsulfamoyl)-N-(3-benzyloxy-7-methoxynaphthyl)glycine methyl ester

To a solution of 1.24 g (8.77 mmol) of chlorosulfonyl isocyanate in 25 mL of methylene chloride is added dropwise a solution of 649 mg (8.76 mmol) of t-butanol in 3 mL methylene chloride. The solution is stirred at room temperature for 30 min then a solution of 2.2 g (6.26 mmol) of (3-benzyloxy-7-methoxynaphthalen-2-ylamino)-acetic acid methyl ester and 1.27 g (12.6 mmol) of triethylamine in 25 mL methylene chloride is added dropwise. The mixture is stirred at room temperature for 90 min then is washed with water. The organic phase is dried over sodium sulfate and the solvent is removed under reduced pressure. The residual oil is purified by flash chromatography using hexane/ethyl acetate (70:30) to elute the title compound as an oil. ¹H-NMR (CDCl₃): δ 8.10 (s, 1H), 7.68 (s, broad, 1H), 7.54 (d, J=9.80 Hz, 1H), 7.49-7.29 (m, 5H), 7.17 (s, 1H), 7.14-7.08 (m, 2H), 5.23 (s, 2H), 4.63 (s, 2H), 3.86 (s, 3H), 3.68 (s, 3H), 1.41 (s, 9H). MS (M−1): 529.

### Step 7

### N-Sulfamoyl-N-(3-benzyloxy-7-methoxynaphthyl)glycine methyl ester

A solution of 3.1 g (5.85 mmol) of N-(t-butoxycarbonylsulfamoyl)-N-(3-benzyloxy-7-methoxynaphthyl)glycine methyl ester in 40 mL trifluoroacetic acid/methylene chloride (1:1) is stirred at room temperature for 30 min. The solvent is removed under reduced pressure. Methylene chloride is added to the residue then is removed under reduced pressure (4×) to give an oily solid which is triturated with ether (16 h) to give the title compound as a solid, mp 137-139°; ¹H-NMR (CDCl₃): δ 8.01 (s, 1H), 7.58 (d, J=8.83 Hz, 1H), 7.51-7.32 (m 5H), 7.23-7.05 (m, 3H), 5.17 (s, broad, 2H), 5.15 (s, 2H), 4.39 (s, 2H), 3.85 (s, 3H), 3.67 (s, 3H). MS (M+1): 431. Anal. Calcd for C₂₁H₂₂N₂O₆S: C, 58.59; H, 5.15; N, 6.51. Found: C, 58.53; H, 5.05; N, 6.62.

### Step 8

### 5-(3-Benzyloxy-7-methoxynaphthalen-2-yl)-1,1-dioxo-[1,2,5]thiadiazolidin-3-one potassium salt

To a solution of 1.89 g (4.39 mmol) of N-sulfamoyl-N-(3-benzyloxy-7-methoxynaphthyl)glycine methyl ester in 5 mL of THF is added dropwise 4.4 mL of a 1.0M solution of potassium t-butoxide in THF. The mixture is stirred at room temperature for 18 h. The resulting thick precipitate is filtered and washed with a small volume of THF. The solid is dried under reduced pressure to give the title compound, mp 182-198°.

### Step 9

### 5-(3-Hydroxy-7-methoxynaphthalen-2-yl)-1,1-dioxo-[1,2,5]thiadiazolidin-3-one potassium salt

A solution of 1.75 g (4.01 mmol) of 5-(3-benzyloxy-7-methoxynaphthalen-2-yl)-1,1-dioxo-[1,2,5]thiadiazolidin-3-one potassium salt in 150 mL of water and is hydrogenated at 50 psi over 500 mg of 10% Pd/C for 24 h. The catalyst is filtered and the aqueous filtrate is washed with ethyl acetate. The aqueous phase is lyophilized to give the title compound as a beige amorphous solid, mp 260-265°. ^{δ 1}H-NMR (DMSO-d6: δ7.90 (s, 1H), 7.54 (d, J=9.04 Hz, 1H), 7.14 (s, 1H), 7.12 (d, J=2.64 Hz, 1H), 6.98 (dd, J=9.04 and 2.64 Hz, 1H), 4.20 (s, 2H), 3.81 (s, 3H). MS (M−1): 307.

### EXAMPLE 2

### 5-(3-Hydroxy-7-propoxynaphthalen-2-yl)-1,1-dioxo-[1,2,5]thiadiazolidin-3-one potassium salt

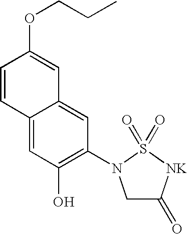

### Step 1

### 3-Hydroxy-7-propoxynaphthalene-2-carboxylic acid propyl ester

To a suspension of 1.6 g (29.6 mmol) of sodium methoxide in DMA (30 mL) is added 3.0 g (14.7 mmol) of 3,7-dihydroxynaphthalene-2-carboxylic acid. The mixture is stirred at room temperature for 1 h then 5.05 g (29.7 mmol) of propyl iodide is added and stirring is continued for 48 h. The mixture is poured into water and acidified with 2N HCl. The mixture is extracted with ethyl acetate and the organic phase is washed with water (3×) and brine (1×). The solution is dried over sodium sulfate and the solvent is removed under reduced pressure. The residual oil is purified by flash chromatography using methylene chloride to elute the title compound which is isolated as a yellow oil. MS (M−1): 287.

### Step 2

### 3-Benzyloxy-7-propoxynaphthalene-2-carboxylic acid propyl ester

To a mixture of 1.5 g (5.21 mmol) of 3-hydroxy-7-propoxynaphthalene-2-carboxylic acid propyl ester and 1.08 g (7.81 mmol) of potassium carbonate in DMF (15 mL) is added 0.98 g (5.73 mmol) of benzyl bromide. The mixture is stirred at room temperature for 48 h then is poured into water. The mixture is extracted with ethyl acetate and the organic phase is washed with water (3×) and brine (1×). The solution is dried over sodium sulfate and the solvent is removed under reduced pressure. The residual oil is purified by flash chromatography using methylene chloride to elute the title compound which is isolated as a yellow oil.

### Step 3

### 3-Benzyloxy-7-propoxynaphthalene-2-carboxylic acid

To a solution of 680 mg (1.8 mmol) of 3-benzyloxy-7-propoxynaphthalene-2-carboxylic acid propyl ester in EtOH (15 mL) is added 2.0 mL of 1.0N NaOH then the mixture is stirred at 60° C. for 3 h. The solvent is removed under reduced pressure and the residual solid is dissolved in water. The solution is washed with MTBE and the aqueous phase is acidified with 1N HCl. The resulting precipitate is filtered, washed with water and dried under reduced pressure to afford the title compound as a white solid, mp 125-128°. MS (M−1): 335.

### 5-(3-Hydroxy-7-propoxynaphthalen-2-yl-1,1-dioxo-[1,2,5]thiadiazolidin-3-one potassium salt

The title compound is prepared from 3-benzyloxy-7-propoxynaphthalene-2-carboxylic acid analogous to Example LBR509 (Steps 3-9), mp 250-255°. ¹H-NMR (DMSO-d6: δ7.88 (s, 1H), 7.54 (d, J=9.04 Hz, 1H), 7.13 (s, 1H), 7.12 (d, J=2.64 Hz, 1H), 6.98 (dd, J=9.04 and 2.64 Hz, 1H), 4.19 (s, 2H), 3.98 (t, 2H), 1.75 (m, 2H), 1.00 (t, 3H). MS (M−1): 335.

### EXAMPLE 3

### 5-(3-Hydroxynaphthalen-2-yl)-1,1-dioxo-[1,2,5]thiadiazolidin-3-one potassium salt

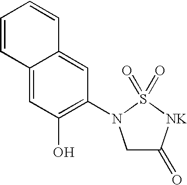

### Step 1

### N-(3-Hydroxynaphthalen-2-yl)-acetamide

To a rapidly stirred mixture of 13.8 g (86.8 mmol) of 3-aminonaphthalen-2-ol, sat. sodium bicarbonate (150 mL) and ether (150 mL) at 0-5° C. is added acetyl chloride (15.8 mL) dropwise and the mixture is stirred at room temperature for 3 h. Any insoluble material is filtered and the aqueous phase is acidified. The mixture is extracted with EtOAc and the organic phase is dried over magnesium sulphate. The solvent is removed under reduced pressure to give the title compound.

### Step 2

### N-(3-Benzyloxynaphthalen-2-yl)-acetamide

To a mixture of 8.8 g (43.8 mmol) of N-(3-hydroxynaphthalen-2-yl)-acetamide and 9.5 g (68.7 mmol) of potassium carbonate in acetone (150 mL) is added 11.5 g (67.3 mmol) of benzyl bromide and the mixture is refluxed for 4 h. The solvent is removed under reduced pressure and water is added to the residue. The mixture is extracted with EtOAc and the organic phase is washed with brine then dried over magnesium sulphate. The solvent is removed under reduced pressure and the residual solid is triturated with ether/hexane (1:2) to afford the title compound.

### Step 3

### 3-Benzyloxynaphthalen-2-ylamine

To 7.1 g (24.4 mmol) of N-(3-benzyloxynaphthalen-2-yl)-acetamide in EtOH (75 mL) is added a solution of KOH in water (10 mL) and the mixture is refluxed for 18 h. Upon cooling, a tan precipitate forms. The solid is filtered and washed with EtOH to give the title compound.

### P 5-(3-Hydroxynaphthalen-2-yl)-1,1-dioxo-[1,2,5]thiadiazolidin-3-one potassium salt

The title compound is prepared from 3-benzyloxynaphthalen-2-ylamine analogous to Example LBR509 (Steps 5-9) using ethyl bromoacetate instead of methyl bromoacetate. ¹H-NMR (DMSO-d6): δ7.73 (s, 1H), 7.43 (d, J=8.08 Hz, 1H), 7.35 (d, J=8.08 Hz, 1H), 7.06 (t, J=7.33 Hz, 1H), 6.96 (t, J=7.20 Hz, 1H), 6.89 (s, broad, 1H), 4.06 (s, 2H). MS (M-1): 277.

### EXAMPLE 4

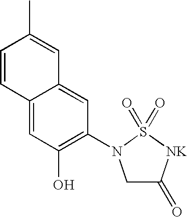

1. (3-Benzyloxy-7-bromonaphthalen-2-yl)-carbamic acid tert-butyl ester (3-Benzyloxy-7-bromonaphthalen-2-yl)-carbamic acid tert-butyl ester is prepared analogously to Example 1, steps 1-3.

2. [(3-Benzyloxy-7-bromonaphthalen-2-yl)-tert-butoxycarbony-amino]-acetic acid methyl ester

To a solution of (3-benzyloxy-bromonaphthalen-2-yl)-carbamic acid tert-butyl ester (38.65 g, 90.2 mmol) in DMF (300 mL) at 0° C. is added NaH (3.79 g, 99.3 mmol). To the solution is added methyl bromoacetate (10.3 mL, 108.2 mmol). The mixture is stirred for 10 min and then quenched with 1N HCl. The solution is extracted with EtOAc and washed with 1N HCl (3×) and sat. NaCl. The organic layer is dried over Na₂SO₄, filtered and concentrated. The residue is recrystallized from EtOAc to afford [(3-benzyloxy-7-bromonaphthalen-2-yl)-tert-butoxycarbony-amino]-acetic acid methyl ester.

3. (3-Benzyloxy-7-bromonaphthalen-2-ylamino)-acetic acid methyl ester

(3-Benzyloxy-7-bromonaphthalen-2-ylamino)-acetic acid methyl ester is prepared analogously to Example 1, steps 2-5.

4. 5-(3-Benzyloxy-7-bromonaphthalen-2-yl)-1,1-dioxo-1,2,5-thiadiazolidin-3-one 5-(3-Benzyloxy-7-bromonaphthalen-2-yl)-1,1-dioxo-1,2,5-thiadiazolidin-3-one is prepared analogously to Example 1, steps 6-8.

5. 5-(3-Benzyloxy-7-methyl-naphthalen-2-yl)-1,1-dioxo-1,2,5-thiadiazolidin-3-one 5-(3-Benzyloxy-7-bromonaphthalen-2-yl)-1,1-dioxo-1,2,5-thiadiazolidin-3-one (4.47 g, 10 mmol), methylboronic acid (1.19 g, 20 mmol), palladium acetate (78 mg, 0.347 mmol), 2-dicyclohexylphosphino-2′,6′-dimethoxybiphenyl, sodium carbonate (30 mL of 2N, 60 mmol.), tetrahydrofuran (100 mL), and dimethoxyethane (100 mL) is heated at 80° C. for 3 days. The mixture is concentrated and extracted between water and diethyl ether. The organic layer is washed with 1:1 water: saturated sodium bicarbonate. The combined aqueous layers are acidified and extracted twice with ethyl acetate. The organic layer is dried over sodium sulfate, filtered and taken directly to the next step.

6. 5-(3-Hydroxy-7-methyl-naphthalen-2-yl)-1,1-dioxo-1,2,5-thiadiazolidin-3-one potassium salt

A solution of 5-(3-Benzyloxy-7-methyl-naphthalen-2-yl)-1,1-dioxo-1,2,5-thiadiazolidin-3-one in 300 mL ethyl acetate from the previous reaction is added to a suspension of Pd(OH)₂/C (0.7 g) in 50 mL EtOH. The resulting suspension is stirred under H₂ for 2d. Celite (2 g) is added and the suspension is filtered through a pad of Celite (2 g) and concentrated. The residue is purified by C18 chromatography on a Biotage 40+M cartridge with a gradient of 0-25% acetonitrile/water at 40 ml/min. The solvent is evaporated to afford the free form of the title compound (1.04 g, 3.56 mmol) that is treated with potassium bicarbonate (7.12 mL of 0.5 M, 3.56 mmol) and concentrated to afford the title compound. ¹H-NMR (MeOD-d3) δ 8.45 (s, 1H), 8.09 (d, J=Hz, 1H), 7.78 (d, J=Hz, 1H), 7.72 (s, 1H), 5.01 (s, 2H), 2.99 (s, 3H). MS (M−1): 291.

### Results

The inhibitory activities (IC₅₀ values) of the compounds to were found to be between 5 nM and 300 nM. The IC₅₀ values were determined using the described assays.

### EQUIVALENTS

Those skilled in the art will recognize, or be able to ascertain using no more than routine experimentation, numerous equivalents to the specific procedures described herein. Such equivalents are considered to be within the scope of the present invention and are covered by the following claims. The appropriate components, processes, and methods of those patents, applications and other documents may be selected for the present invention and embodiments thereof.

What is claimed is:

1. A compound, which is selected from the group consisting of
5-(3-Hydroxy-7-methoxynaphthalen-2-yl)-1,1-dioxo-[1,2,5]thiadiazolidin-3-one;
5-(3-Hydroxy-7-propoxynaphthalen-2-yl)-1,1-dioxo-[1,2,5]thiadiazolidin-3-one;
5-(3-Hydroxynaphthalen-2-yl)-1,1-dioxo-[1,2,5]thiadiazolidin-3-one; and
5-(3-Hydroxy-7-methyl-naphthalen-2-yl)-1,1-dioxo-1,2,5-thiadiazolidin-3-one;
or a pharmaceutically acceptable salt thereof.

2. The compound of claim 1, wherein the compound is selected from group consisting of
5-(3-Hydroxy-7-methoxynaphthalen-2-yl)-1,1-dioxo-[1,2,5]thiadiazolidin-3-one potassium salt;
5-(3-Hydroxy-7-propoxynaphthalen-2-yl)-1,1-dioxo-[1,2,5]thiadiazolidin-3-one potassium salt;
5-(3-Hydroxynaphthalen-2-yl)-1,1-dioxo-[1,2,5]thiadiazolidin-3-one potassium salt; and
5-(3-Hydroxy-7-methyl-naphthalen-2-yl)-1,1-dioxo-1,2,5-thiadiazolidin-3-one potassium salt.

3. A method for the inhibition of PTPase activity, comprising:
administering to a mammal in need thereof a therapeutically effective amount of a compound of claim 1, or a pharmaceutically acceptable salt thereof.

4. A method for the treatment of conditions mediated by PTPase activity, comprising:
administering to a mammal in need thereof a therapeutically effective amount of a compound of claim 1, or a pharmaceutically acceptable salt thereof.

5. The method according to claim 3, which method comprises administering a therapeutically effective amount of a combination of said compound, or a pharmaceutically acceptable salt thereof, and an anti-diabetic agent, a hypolipidemic agent, an anti-obesity agent or an anti-hypertensive agent.

6. A method for the treatment of conditions mediated by PTP-1B activity in mammals comprising:
administering to a mammal in need thereof a therapeutically effective amount of a compound of claim 1, or a pharmaceutically acceptable salt thereof.

7. A method for modulating glucose levels in mammals, comprising:
administering to a mammal in need thereof a therapeutically effective amount of a compound of claim 1, or a pharmaceutically acceptable salt thereof.

8. A method for the treatment of insulin resistance, glucose intolerance, type 2 diabetes, diabetic renal insufficiency, non-diabetic renal insufficiency, diabetic nephropathy, glomerulonephritis, glomerular sclerosis, proteinuria of primary renal disease, diabetic retinopathy, obesity, acute congestive heart failure, chronic congestive heart failure, left ventricular dysfunction, hypertrophic cardiomyopathy, diabetic cardiac myopathy, supraventricular arrhythmias, ventricular arrhythmias, atrial fibrillation, atrial flutter, hypertension, primary pulmonary hypertension, secondary pulmonary hypertension, renal vascular hypertension, dyslipidemia, atherosclerosis, ischemic diseases of the large or small blood vessels, angina pectoris, myocardial infarction, ischemia/reperfusion injury, detrimental vascular remodeling, vascular restenosis, migraine, peripheral vascular disease, and Raynaud's disease, irritable bowel syndrome, pancreatitis, cancer, osteoporosis, multiple sclerosis, stroke, spinal cord injury, Alzheimer's disease, Parkinson's disease, Huntington's disease, spinocerebellar ataxia, infectious diseases, inflammation, or muscle degeneration disorders comprising:
administering to a mammal in need thereof a therapeutically effective amount of a compound of claim 1, or a pharmaceutically acceptable salt thereof.

9. A pharmaceutical composition, comprising:
a compound according to claim 1, or a pharmaceutically acceptable salt thereof, and
one or more pharmaceutically acceptable carriers.

10. The pharmaceutical composition according to claim 9, further comprising at least one additional anti-diabetic agent, hypolipidemic agent, anti-obesity agent or anti-hypertensive agent.

11-14. (canceled)
